(19) United States
(12) Patent Application Publication
(10) Pub. No.: US 2022/0000795 A1
(43) Pub. Date: Jan. 6, 2022

(54) Water Soluble Cannabinoid Product with Reduced Particle Size for Enhanced Delivery

(71) Applicant: Centuria Foods, Inc., Carson City, NV (US)

(72) Inventors: Kevin Schmidt, Reno, NV (US); Jason Hastings, Carson City, NV (US)

(73) Assignee: Centuria Foods, Inc., Carson City, NV (US)

(21) Appl. No.: 16/921,010

(22) Filed: Jul. 6, 2020

(51) Int. Cl. A61K 31/05; A61K 47/46; A61K 47/44; A61K 47/02; A61K 47/12; A61K 9/107

(52) U.S. Cl. CPC A61K 31/05; A61K 47/46; A61K 9/107; A61K 47/02; A61K 47/12; A61K 47/44

(57) ABSTRACT

An aqueous cannabinoid product including surfactant, preservative, acidulant, and cosurfactant. Most commonly cannabidiol is the cannabinoid product in the aqueous product. The cosurfactant used is typically a phytochemical product which reduces the particle size of the aqueous cannabinoid emulsion.

### BACKGROUND

### Field of the Invention

The present invention relates generally to health products. More specifically, the present invention relates to water soluble cannabinoid products for oral consumption.

### Description of the Related Art

Most cannabinoid products currently available for oral consumption entail oils to incorporate the hydrophobic nature of the cannabinoids. Due to the nature of the human body, oils suffer from low bioavailability across the intestinal membrane. The low bioavailability reduces the effects that the cannabinoid has on the body or requires additional consumption. This is wasteful and can be expensive.

The currently available aqueous cannabinoid products for oral consumption are largely plagued by low bioavailability of the cannabinoid due to large particle sizes. Emulsion products are becoming more common to address the poor solubility of the cannabinoid, but still suffer from low bioavailability. Using excipients to decrease the particle size of a solubilized or emulsified cannabinoid is not common in the industry.

### SUMMARY

In accordance with the embodiments herein, a product is described containing a cannabinoid and an excipient to decrease the particle size of the cannabinoid emulsion between 5 percent and 15 percent for every 0.1 percent cosurfactant used. This invention pertains to cannabinoid emulsions having an average particle size of less than 350 nm in diameter. Generally, the product contains the following six ingredients i) water; ii) cannabinoid; iii) surfactant; iv) preservative; v) acidulant; vi) and cosurfactant to reduce the average particle size of the emulsion.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

In an illustrative embodiment of the invention, the product is generally composed of the following six ingredients i) water; ii) cannabinoid; iii) surfactant; iv) preservative; v) acidulant; vi) and cosurfactant to reduce the average particle size of the emulsion. Typically, the water in the product makes up from 25.0 to 70.0 percent of the product by mass.

In further embodiments, the cannabinoid may be from 2.0 to 25.0 percent of the product by mass. Additionally, the cannabinoid is typically cannabidiol, but other cannabinoids are contemplated.

The surfactant, in other embodiments, may be from 5.0 to 25.0 percent of the product by mass. The surfactant may be PEG-40, lecithin, or quillaja saponin. The PEG-40 used may be hydrogenated or non-hydrogenated castor oil. Other surfactants are contemplated.

In other embodiments, the preservative is from 0.08 to 0.20 percent of the product by mass. Typically, potassium-based preservatives, such as potassium sorbate and potassium benzoate, are used in the product. Other preservatives are contemplated. Some embodiments use multiple preservatives, such as both potassium sorbate and potassium benzoate.

In further embodiments, the acidulant may be citric acid. However, other acidulants are contemplated. Typically, the acidulant is no more than 0.05 percent of the mass of the product.

The cosurfactant, in further embodiments, may be a phytochemical. Other cosurfactants are contemplated. Usually, the cosurfactant is from 0.1 to 5.0 percent of the product by mass. The use of the cosurfactant reduces the particle size of the emulsion between 5% and 15% per 0.1% cosurfactant.

Some embodiments also include an emulsifier, such as polysorbates, sorbitan monolaurate, lecithins, or other known emulsifiers. The emulsifier may be from 5.0 to 25.0 percent of the product by mass.

Other embodiments may also include a sweetener, such as sugar or sugar substitutes. Sugar substitutes such as acesulfame potassium, agave nectar, aspartame, corn syrup, glucose syrup, glycerol, golden syrup, honey, high-fructose corn syrup, mogrosides, saccharin, stevia, sucralose, and sugar alcohols are contemplated.

In one example embodiment, the product may be a solution of 39.8 percent water, 20.0 percent cannabidiol, 20.0 percent quillaja saponin, 2.0 percent phytochemical, 18.0 percent fractionated coconut oil, 0.08 percent potassium sorbate, 0.08 percent potassium benzoate, and 0.04 percent citric acid. All percentages in the forgoing example embodiment are by mass.

Another example embodiment is the product as a solution of 66.3 percent water, 25.0 percent PEG-40, 5.0 percent phytochemical, 3.0 percent cannabidiol, 0.5 percent stevia, 0.08 percent potassium sorbate, 0.08 percent potassium benzoate, and 0.04 percent citric acid. All percentages are by mass in the forgoing example embodiment. All percentages in the forgoing example embodiment are by mass.

What is claimed is:

1. A cannabinoid product, comprising:
water;
at least one emulsified cannabinoid;
at least one surfactant;
at least one preservative;
at least one acidulant; and
a cosurfactant;
wherein the cosurfactant reduces the particle size of the emulsified cannabinoid.

2. The cannabinoid product of claim 1, wherein the at least one emulsified cannabinoid is cannabidiol.

3. The cannabinoid product of claim 1, wherein the at least one surfactant is selected from the group consisting of PEG-40 hydrogenated castor oil, PEG-40 non-hydrogenated castor oil, lecithin, and quillaja saponin.

4. The cannabinoid product of claim 1, wherein the at least one preservative is at least one potassium-based preservative.

5. The cannabinoid product of claim 5, wherein the at least one potassium-based preservative is selected from the group consisting of potassium sorbate and potassium benzoate.

6. The cannabinoid product of claim 1, wherein the at least one acidulant is citric acid.

7. The cannabinoid product of claim 1, wherein the cosurfactant is a phytochemical.

8. The cannabinoid product of claim 1, wherein the cosurfactant reduces the particle size of the emulsified cannabinoid from 5.0 to 15.0 percent per 0.1% of the cosurfactant used.

9. The cannabinoid product of claim 1, wherein the water is from 25.0 to 70.0 percent by mass of the product.

10. The cannabinoid product of claim 1, wherein the emulsified cannabinoid is from 2.0 to 25.0 percent by mass of the product.

11. The cannabinoid product of claim 1, wherein the surfactant is from 5.0 to 25.0 percent by mass of the product.

12. The cannabinoid product of claim 1, wherein the preservative is from 0.08 to 0.20 percent by mass of the product.

13. The cannabinoid product of claim 1, wherein the acidulant is no more than 0.05 percent by mass of the product.

14. The cannabinoid product of claim 1, wherein the cosurfactant is from 0.5 to 5.0 percent by mass of the product.

15. The cannabinoid product of claim 1, further comprising an emulsifier.

16. The cannabinoid product of claim 15, wherein the at least one emulsifier is selected from the group consisting of polysorbates, sorbitan monolaurate, lecithins, and other known emulsifiers.

17. The cannabinoid product of claim 15, wherein the emulsifier is from 5.0 to 25.0 percent by mass of the product.

18. The cannabinoid product of claim 1, further comprising a sweetener selected from the group consisting of sugar, acesulfame potassium, agave nectar, aspartame, corn syrup, glucose syrup, glycerol, golden syrup, honey, high-fructose corn syrup, mogrosides, saccharin, stevia, sucralose, and sugar alcohols.

19. A cannabinoid product, comprising:
water at 39.8 percent by mass;
cannabidiol at 20.0 percent by mass;
quillaja saponin at 20.0 percent by mass;
a phytochemical product at 2.0 percent by mass;
fractionated coconut oil at 18.0 percent by mass;
potassium sorbate at 0.08 percent by mass;
potassium benzoate at 0.08 percent by mass; and
citric acid at 0.04 percent by mass;
wherein the phytochemical product reduces the particle size of the emulsion.

20. A cannabinoid product, comprising:
water at 66.3 percent by mass;
PEG-40 at 25.0 percent by mass;
a phytochemical product at 5.0 percent by mass;
cannabidiol at 3.0 percent by mass;
stevia at 0.5 percent by mass;
potassium sorbate at 0.08 percent by mass;
potassium benzoate at 0.08 percent by mass; and
citric acid at 0.04 percent by mass;
wherein the phytochemical product reduces the particle size of the emulsion.
